(19) United States
(12) Patent Application Publication
(10) Pub. No.: US 2023/0107315 A1
(43) Pub. Date: Apr. 6, 2023

(54) LEVELS OF IMMUNE RESPONSE MARKERS AS ADVERSE OUTCOME PREDICTORS FOLLOWING BIOMATERIAL IMPLANT SURGERY

(71) Applicant: UNIVERSITY OF SOUTH CAROLINA, Colombia, SC (US)

(72) Inventors: MELISSA MOSS, COLUMBIA, SC (US); MIHYUN L. WAUGH, CAYCE, SC (US); NICHOLAS D. BOLTIN, COLUMBIA, SC (US); LAUREN WOLF, CAMBRIDGE, MA (US); RONNIE HORNER, OMAHA, NE (US); MATTHEW HERMES, SIMPSONVILLE, SC (US); THOMAS WHEELER, GREENVILLE, SC (US); RICHARD GOODWIN, GREENVILLE, SC (US); RICHARD MICHAEL GOWER, COLUMBIA, SC (US)

(21) Appl. No.: 17/960,462

(22) Filed: Oct. 5, 2022

Related U.S. Application Data
(60) Provisional application No. 63/252,406, filed on Oct. 5, 2021.

(51) Int. Cl. G16H 50/30; G16B 5/00; G16B 40/20

(52) U.S. Cl. CPC G16H 50/30; G16B 5/00; G16B 40/20

(57) ABSTRACT

In general, the present disclosure is directed to systems and methods of evaluating a subject's risk of one or more complications associated with pelvic organ prolapse surgery. The method comprising: obtaining, by a computing system comprising one or more computing devices, sample data associated with the subject; inputting, by the computing system, the sample data into a machine-learned immune response model; receiving, by the computing system as an output of the machine-learned immune response model, one or more predictions of post-surgical complications of mesh exposure through the vaginal wall associated with the subject; and performing a pelvic organ prolapse repair surgery on the subject, wherein the surgery is performed based at least in part on the one or more predictions of post-surgical complications by the machine-learned immune response model associated with a likelihood of success.

### CROSS REFERENCE TO RELATED APPLICATION

The present application claims priority to U.S. Provisional Patent Application Serial No. 63/252,406, having a filing date of Oct. 5, 2021, entitled “Blood-Cytokine Levels as Adverse Outcome Predictors Following Biomaterial Implant Surgery,” which is incorporated herein by reference in its entirety.

### BACKGROUND

Pelvic organ prolapse (POP), defined as symptomatic descent of the vaginal wall, is caused from vaginal births, obesity, and age. This widespread condition affects about 50% of parous women and 6% of non-parous women between the ages of 20 and 59. Standard treatment has included surgical intervention, which can be augmented via implantation of polypropylene vaginal mesh to provide mechanical support and reinforcement of the prolapsed organ. Unfortunately, post-surgical complications, predominantly mesh exposure into the vaginal wall, result in chronic pain and decreased quality of life. Consequently, patients can be left with residual symptoms and emotional distress. Some patients elect for surgical reintervention to revise or remove the mesh implantation. Indeed, 37 out of 482 patients (7.9%) underwent further surgery to remove the mesh, and 7 patients (1.8%) repeated the prolapse surgery. Due to these complications, transvaginal mesh kits were pulled from the market by the FDA in 2019. A clinical decision support tool that could better inform both patients and surgeons about the risk of complications following pelvic organ prolapse surgery could allow for reintroduction of this advantageous surgical augmentation.

### SUMMARY

In general, the present disclosure is directed to methods of evaluating a subject's risk of one or more complications associated with pelvic organ prolapse surgery, the method comprising: 1) obtaining, by a computing system comprising one or more computing devices, sample data associated with the subject; 2) inputting, by the computing system, the sample data into a machine-learned immune response model; 3) receiving, by the computing system as an output of the machine-learned immune response model, one or more predictions of post-surgical complications of mesh exposure through the vaginal wall associated with the subject; and 4) performing a pelvic organ prolapse repair surgery on the subject wherein the surgery is performed based, at least in part, on the one or more predictions of post-surgical complications by the machine-learned immune response model associated with a likelihood of success.

Also, the present disclosure is directed to a computing system. The computing system comprising: 1) a machine-learned immune response model trained with training data; 2) one or more processors; and 3) one or more non-transitory computer-readable media that store instructions that, when executed by the one or more processors, cause the one or more processors to perform operations. According to the present disclosure, the operations comprising: 1) obtaining sample data associated with a subject, wherein the data comprises at least one set of sample data associated with the subject; 2) inputting the sample data into the machine-learned immune response model; and 3) receiving, as an output of the machine-learned immune response model, one or more predicted surgical outcomes associated with the subject.

Through these and other features and aspects, embodiments and advantages of the present invention will become better understood with reference to the following description and appended claims.

### BRIEF DESCRIPTION OF THE FIGURES

A full and enabling disclosure of the present disclosure is set forth more particularly in the remainder of the specification, including reference to the accompanying figures, in which:

FIG. 1 depicts an example processing workflow for evaluating a subject's risk of post-surgical complications arising from pelvic organ prolapse surgery.

FIG. 2 depicts an example workflow for training an immune response model.

FIG. 3 depicts an example computing system 100 for machine-learning-based analysis of immune response markers.

FIG. 4A depicts bi-plot of blood samples comprising immune response markers incubated with inflammatory agent lipopolysaccharide (LPS).

FIG. 4B depicts bi-plot of blood samples comprising immune response markers incubated with polypropylene mesh.

FIG. 4C depicts bi-plot of blood samples comprising immune response markers incubated alone.

FIG. 5A depicts immune response markers contribution to PC 1.

FIG. 5B depicts immune response markers contribution to PC 2.

FIG. 6 depicts bi-plot of subject's test sample averages (indicated by numbers) exhibiting the presence or absence of vaginal mesh exposure.

FIG. 7A depicts contribution of individual immune response markers based on Artificial Neural Network.

FIG. 7B depicts contribution of individual immune response markers based on Decision Tree.

FIG. 7C depicts contribution of individual immune response markers based on Naive Bayes.

FIG. 7D depicts contribution of individual immune response markers based on Logistic Regression.

Repeat use of reference characters in the present specification and drawings is intended to represent the same or analogous features or elements of the present disclosure.

### DETAILED DESCRIPTION

Reference will now be made in detail to example embodiments of the disclosure. It is to be understood by one of ordinary skill in the art that the present disclosure is a description of exemplary embodiments only and is not intended as limiting the broader aspects of the present disclosure.

The present disclosure is generally directed to systems and methods that include or otherwise leverage a machine-learned immune response model to predict and/or evaluate a subject's risk of post-surgical complications associated with the subject for mesh exposure through the vaginal wall following pelvic organ prolapse surgery. Advantageously, systems and methods disclosed herein utilize supervised learning models with a high degree of accuracy, specificity, and sensitivity to predict surgical outcomes based on blood immune response marker analysis. For instance, systems and methods disclosed herein may be utilized in a clinical setting to assist clinicians with decisions as to whether they should proceed with pelvic organ prolapse repair surgeries in subjects.

With reference now to the Figures, example embodiments of the present disclosure will be discussed in further detail.

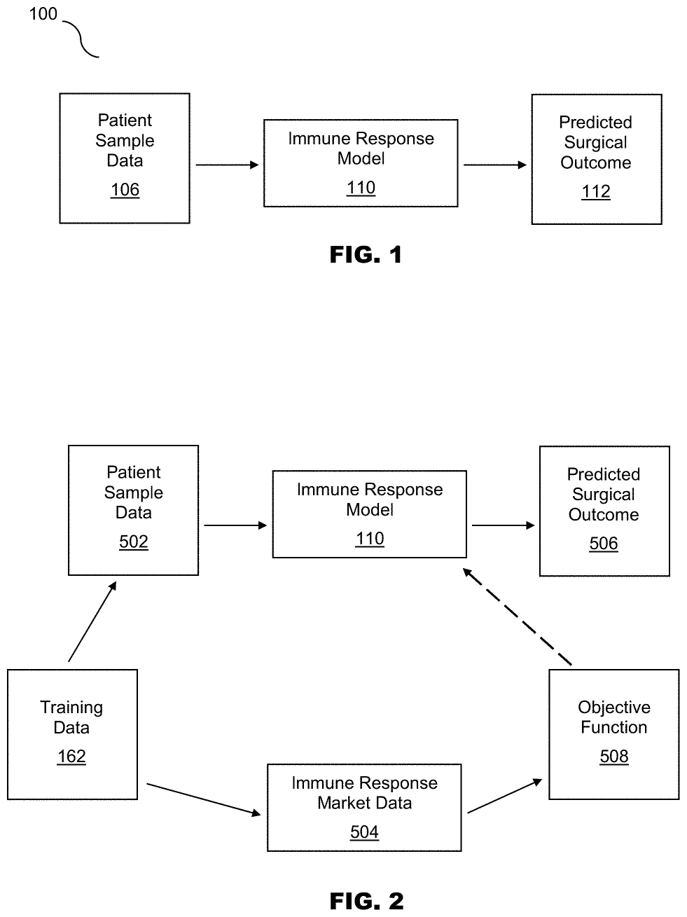

Turning first to FIG. 1, an example processing workflow is provided for evaluating a subject's risk for pelvic organ prolapse surgery success according to example embodiments disclosed herein. In particular, the processing workflow includes, or otherwise leverages, a machine-learned immune response markers expression model.

A clinician may collect and process a biological sample from a subject to classify whether particular immune response markers are expressed at a level in which the subject is likely to have a successful pelvic organ prolapse surgery. In some embodiments, the present disclosure is useful for assessing whether or not a pelvic organ prolapse surgery should be performed on a subject based on levels of immune response markers in their biological sample, prior to or following exposure to an inflammatory agent or a biomaterial, using a statistical algorithm and/or empirical data (e.g., the amount of immune response markers described herein, such as in the tables, figures, examples, and otherwise described herein).

According to the present disclosure, the biological sample collected from a patient may be a blood sample. In some embodiments, a blood sample may be collected from an upper extremity of a subject. The blood sample may be incubated with inflammatory agent lipopolysaccharide (LPS) or sterile polypropylene mesh. Further, the blood sample from a test subject may be collected and mixed with an agent, such as a protein-binding agent like an antibody or antigen-binding fragment thereof, or a nucleic acid-binding agent like an oligonucleotide, capable of detecting the amount of immune response markers in the biological sample. In some embodiments, at least one antibody or antigen-binding fragment thereof is used, wherein two, three, four, five, six, seven, eight, nine, ten, or more such antibodies or antibody fragments can be used in combination (e.g., in multiplex assay) or in serial. In certain instances, the statistical algorithm is a single learning statistical classifier system. For example, a single learning statistical classifier system can be used to classify a sample based on a prediction or probability value and the presence or level of the immune response markers. The use of a single learning statistical classifier system typically classifies the sample as, for example, a likely successful candidate for pelvic organ prolapse repair surgery with a sensitivity, specificity, positive predictive value, negative predictive value, and/or overall accuracy of from about 50% to about 90%, such as at least about 75%, 76%, 77%, 78%, 79%, 80%, 81%, 82%, 83%, 84%, 85%, 86%, 87%, 88%, 89%, or 90%.

Once again turning to FIG. 1, the patient sample data 106 can include immune response marker data based on the blood sample collected from a subject. According to the present disclosure, levels of immune response markers in each blood sample may be quantified using a multiplex assay. According to the present disclosure, immune response markers may include, but are not limited to, pro-inflammatory cytokines, anti-inflammatory cytokines, Treg cytokines, tumor necrosis factor (TNF) superfamily proteins, interferons (IFN) family proteins, T helper 17 (Th17) immunity related genes, and matrix metalloproteinases (MMPs). For instance, immune response markers quantified may include, but are not limited to, IL-1α, IL-1β, IL-2, IL-4, IL-6, IL-8, IL-10, IL-11, IL-12 p40, IL-12 p70, IL-17A, IL-17F, IL-21, IL-19, IL-20, IL-22, IL-23, IL-25, IL-26, IL-27 p28, IL-28A/IFN-I2, IL-29/IFN-I1, IL-31, IL-32, IL-33, IL-34, IL-35, sIL-6Ra, pg130/sIL-6Rβ, IFN-a2, IFN-β, IFN- [symbol] , TNF-α, sTNF-R1, sTNF-R2, TWEAK/TNFSF12, APRIL/TNFSF13, BAFF/TNFSF13B, LIGHT/TNFSF14, TSLP, GM-CSF, sCD40L, sCD163, sCD30/TNFRSF8, Chitinase-3-like 1, MMP-1, MMP-2, MMP-3, Osteocalcin, Osteopontin, Pentraxin-3, or a combination thereof.

The computing system 100 can input the test data 106 into the machine-learned immune response model 110. For example, the machine-learned immune response model 110 may include a machine learning algorithmic technique capable of adapting to complex data sets (e.g., panel of immune response markers of interest) and making decisions based upon such data sets. Examples the machine-learned immune response model 110 may be learning statistical classifier systems include, but are not limited to, those using inductive learning (e.g., decision/classification trees such as random forests, classification and regression trees (C&RT), boosted trees, etc.); Probably Approximately Correct (PAC) learning; connectionist learning (e.g., neural networks (NN); artificial neural networks (ANN); neuro fuzzy networks (NFN); network structures; perceptrons such as multi-layer perceptrons; multi-layer feed-forward networks; applications of neural networks; Bayesian learning in belief networks, etc.); reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In some embodiments, the machine-learned immune response model 110 may include a deep artificial neural network, a support vector machine, a decision tree, a naïve bayes, and/or a logistic regression.

The machine-learned immune response model 110 and/or associated computing system components may output or otherwise generate and provide a visualization of the one or more immune markers expression patterns in the blood sample. Output data may reflect the concentration of immune markers expressed in the blood sample. For instance, the output of the machine-learned immune response model 110 can predict post-surgical complications arising from a pelvic organ prolapse repair surgery performed on a subject based on levels of immune response markers. Thus, the model 110 can predict surgical outcome 112 based on levels of immune response markers, thereby providing a treatment guide for clinicians (e.g., surgeons).

Thus, in some implementations, the computing system 100 can take biological testing data as input and, in response, provide the clinician a prediction as to whether the post-surgical mesh exposure into the vaginal wall is likely to occur based on pre-determined levels of immune markers. One benefit of the described system is that it can guide the treatment expectations for people with pelvic organ prolapse prior to surgery.

FIG. 2 depicts an example workflow for training an immune response model 110 according to embodiments disclosed herein. For example, the workflow may be performed by a machine-learning computing system 100 (e.g., the model training 160 of machine-learning computing system 140, as described herein).

In some embodiments, the machine-learned immune response model 110 can be trained on training data 162. The training data 162 may include sets of sample data 502 that are labeled with a plurality of immune response data 504. That is, each set of sample data 502 may have an associated immune marker level data 504 that describes the expression level of an immune response marker or immune response markers associated with a subject that generated the corresponding sample data 502.

According to the present disclosure, principal component analysis may be used to gain insight into levels of immune markers expression. “Principal component analysis” (PCA) herein refers to a statistical procedure that uses an orthogonal transformation to convert a set of observations of variables into a set of values of linearly uncorrelated variables called principal components (PC). The transformation is defined in such a way that the first principal component (PC 1) has the largest possible variance and each succeeding component, in turn, has the highest variance possible under the constraint that it is orthogonal to the preceding components (e.g., PC 2). Each PC can be a feature or all PCs concatenated together can be a feature.

Data analyzed by PCA may be utilized to uncover associations among immune response markers and identify immune response marker patterns that correlate with post-surgical mesh exposure into the vaginal wall. For instance, immune response markers that contribute most to each PC may be divided into pro-inflammatory (PC 1) and anti-inflammatory (PC 2) cytokines. In one embodiment, pro-inflammatory cytokines, such as IL-12 p40, IL-1α, and TNF-α, juxtaposed with PC values that correspond to post-surgical mesh exposure, suggesting these cytokines are associated with the presence of post-surgical mesh exposure. These observations may inspire potential therapeutic strategies that could improve post-surgical outcomes. For example, the surgical mesh could be designed to modulate these key pro-inflammatory cytokines. In this way, while supporting the pelvic structure, the mesh could simultaneously function in controlling the immune response to minimize biomaterial rejection.

According to the present disclosure, supervised machine learning models may predict the presence or absence of post-surgical mesh exposure. Levels of immune markers in patients with known surgical outcomes can be used to train and test these models, as well as probe the number of immune markers required for effective predictions. In one embodiment, various machine learning algorithms are used to demonstrate predictive capabilities of a group of 13 immune markers in a small group of subjects, with prediction accuracy ranging from about 50% to about 90%. Interestingly, reducing the number of immune markers from 13 to 7 reduced prediction accuracy by less than 10%. Interestingly, Logistic Regression actually increased prediction accuracy versus the larger group of immune response markers.

Each set of sample data 502 may be input into the immune response model 110. In response, the immune response model 100 can output one or more predicted surgical outcomes 506 based on the plurality of immune response markers present in each set of sample data 502. An objective function 508 can evaluate a difference between the immune response model 100 for each set of sample data 502 and the levels of immune marker(s) data 504 associated with such set of test data 502.

FIG. 2 illustrates one example supervised learning workflow. Other training techniques can be used in addition, or alternatively, to the example workflow shown in FIG. 2.

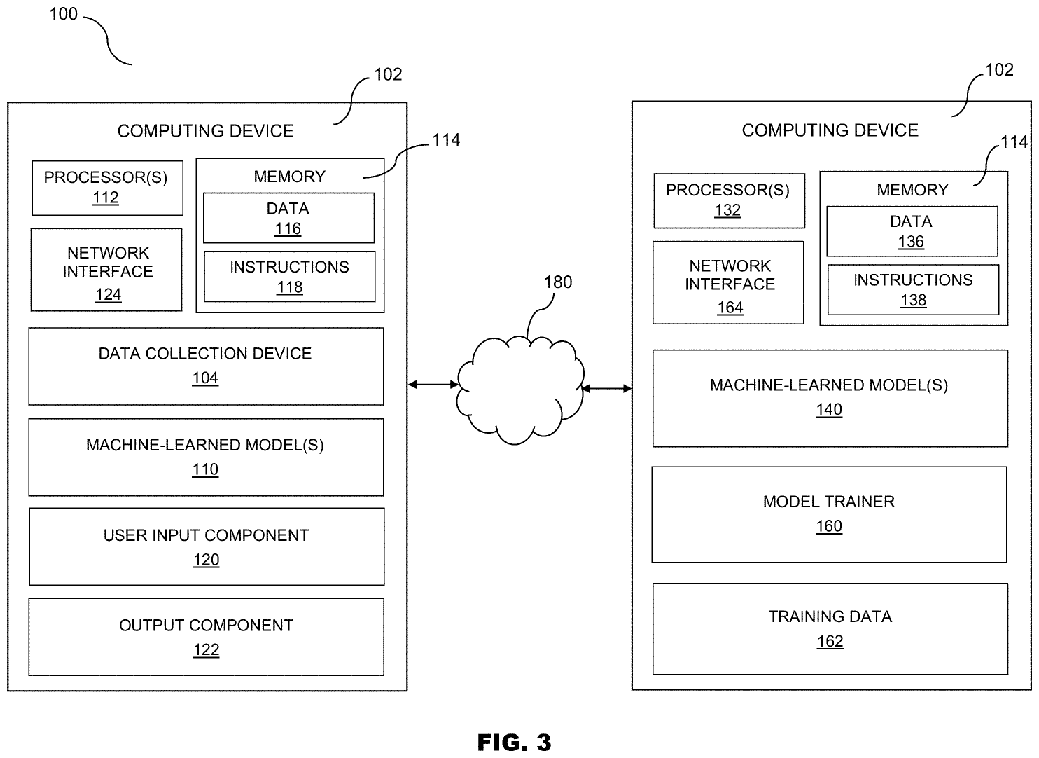

FIG. 3 depicts an example computing system 100 for machine-learning-based analysis of immune response markers according to example embodiments of the present disclosure. The example system 100 includes a computing device 102 and a machine learning computing system 130 that are communicatively coupled over a network 180.

The computing device 102 includes one or more processors 112 and a memory 114. The one or more processors 112 can be any suitable processing device (e.g., a processor core, a microprocessor, an ASIC, a FPGA, a controller, a microcontroller, etc.) and can be one processor or a plurality of processors that are operatively connected. The memory 114 can include one or more non-transitory computer-readable storage media, such as RAM, ROM, EEPROM, EPROM, one or more memory devices, flash memory devices, etc., and combinations thereof.

The memory 114 can store information that can be accessed by the one or more processors 112. For instance, the memory 114 (e.g., one or more non-transitory computer-readable storage mediums, memory devices, etc.) can store data 116 that can be obtained, received, accessed, written, manipulated, created, and/or stored. In some implementations, the computing device 102 can obtain data from one or more memory device(s) that are remote from the device 102.

The memory 114 can also store computer-readable instructions 118 that can be executed by the one or more processors 112. The instructions 118 can be software written in any suitable programming language or can be implemented in hardware. Additionally, or alternatively, the instructions 118 can be executed in logically and/or virtually separate threads on processor(s) 112.

For example, the memory 114 can store instructions 118 that, when executed by the one or more processors 112, cause the one or more processors 112 to perform any of the operations and/or functions described herein.

According to an aspect of the present disclosure, the computing device 102 can store or include one or more machine-learned models 110. For example, the models 100 can be or can otherwise include various machine-learned models such as a random forest classifier; a logistic regression classifier; a support vector machine; one or more decision trees; a neural network; and/or other types of models including both linear models and non-linear models. Example neural networks include feed-forward neural networks, recurrent neural networks (e.g., long short-term memory recurrent neural networks), convolutional neural networks, or other forms of neural networks.

In some implementations, the computing device 102 can receive the one or more machine-learned models 100 from the machine learning computing system 130 over network 180 and can store the one or more machine-learned models 100 in the memory 114. The computing device 102 can then use or otherwise run the one or more machine-learned models 100 (e.g., by processor(s) 112).

The machine learning computing system 130 includes one or more processors 132 and a memory 134. The one or more processors 132 can be any suitable processing device (e.g., a processor core, a microprocessor, an ASIC, a FPGA, a controller, a microcontroller, etc.) and can be one processor or a plurality of processors that are operatively connected. The memory 134 can include one or more non-transitory computer-readable storage media, such as RAM, ROM, EEPROM, EPROM, one or more memory devices, flash memory devices, etc., and combinations thereof.

The memory 134 can store information that can be accessed by the one or more processors 132. For instance, the memory 134 (e.g., one or more non-transitory computer-readable storage mediums, memory devices) can store data 136 that can be obtained, received, accessed, written, manipulated, created, and/or stored. In some implementations, the machine learning computing system 130 can obtain data from one or more memory device(s) that are remote from the system 130.

The memory 134 can also store computer-readable instructions 138 that can be executed by the one or more processors 132. The instructions 138 can be software written in any suitable programming language or can be implemented in hardware. Additionally, or alternatively, the instructions 138 can be executed in logically and/or virtually separate threads on processor(s) 132.

For example, the memory 134 can store instructions 138 that, when executed by the one or more processors 132, cause the one or more processors 132 to perform any of the operations and/or functions described herein.

In some implementations, the machine learning computing system 130 includes one or more server computing devices. If the machine learning computing system 130 includes multiple server computing devices, such server computing devices can operate according to various computing architectures, including, for example, sequential computing architectures, parallel computing architectures, or some combination thereof.

In addition, or alternatively, to the model(s) 100 at the computing device 102, the machine learning computing system 130 can include one or more machine-learned models 140. For example, the models 140 can be or can otherwise include various machine-learned models such as a random forest classifier; a logistic regression classifier; a support vector machine; one or more decision trees; a neural network; and/or other types of models including both linear models and non-linear models. Example neural networks include feed-forward neural networks, recurrent neural networks (e.g., long short-term memory recurrent neural networks), convolutional neural networks, or other forms of neural networks.

Thus, machine-learned models 100 can be located and used at the computing device 102 and/or machine-learned models 140 can be located and used at the machine learning computing system 130.

In some implementations, the machine learning computing system 130 and/or the computing device 102 can train the machine-learned models 100 and/or 140 through use of a model trainer 160. The model trainer 160 can train the machine-learned models 100 and/or 140 using one or more training or learning algorithms. One example training technique is backwards propagation of errors (“backpropagation”).

In some implementations, the model trainer 160 can perform supervised training techniques using a set of labeled training data 162, for example, as described with reference to FIG. 2. In other implementations, the model trainer 160 can perform unsupervised training techniques using a set of unlabeled training data. The model trainer 160 can perform a number of generalization techniques to improve the generalization capability of the models being trained. Generalization techniques include weight decays, dropouts, or other techniques. The model trainer 160 can be implemented in hardware, software, firmware, or combinations thereof.

The computing device 102 can also include a network interface 124 used to communicate with one or more systems or devices, including systems or devices that are remotely located from the computing device 102. The network interface 124 can include any circuits, components, software, etc. for communicating with one or more networks (e.g., 180). In some implementations, the network interface 124 can include, for example, one or more of a communications controller, receiver, transceiver, transmitter, port, conductors, software and/or hardware for communicating data. Similarly, the machine learning computing system 130 can include a network interface 164.

Certain aspects of the present disclosure may be better understood according to the following examples, which are intended to be non-limiting and exemplary in nature. Moreover, it will be understood that the compositions described in the examples may be substantially free of any substance not expressly described.

### EXAMPLES

### Methods

### Study Population

Twenty healthy, non-pregnant female subjects between the age of 56-89 years with a history of surgical intervention to correct POP via a procedure that utilized polypropylene mesh were selected for the study. These subjects included ten that experienced post-surgical exposure of the mesh through the vaginal wall and ten that did not. Subjects with POP relapse and/or were currently on medications that would alter inflammatory response were excluded from this study. All samples collected and data analyzed were de-identified and followed IRB protocol.

### Blood Sample Collection and Processing

Blood samples were obtained from the 20 selected subjects. Approximately 12 mL of blood was drawn from an upper extremity of each subject into three BD Vacutainer® EDTA-coated tubes. De-identified blood samples were then transferred to a lab facility for immediate processing. Each subject's blood sample was divided into equal aliquots for 24-h incubation at 37° C. under three distinct conditions: 1) incubation with inflammatory agent lipopolysaccharide (LPS) at 20 ng/ml (positive control); 2) incubation with sterile polypropylene mesh area of 2 cm×2 cm (experimental); and 3) incubation alone (negative control). After incubation, the plasma layer was collected following centrifugation (1500×g, 10 min, 4° C.) and immediately stored at −80° C.

### Measurement of Blood Immune Marker Levels

As markers of immune response, cytokine levels in each blood sample were quantified using the bead-based MILLIPLEX® Human Cytokine/Chemokine/Growth Factor Panel A—Immunology Multiplex Assay (EMD Millipore Corporation, Billerica, Mass.), which is comprised of analytes for target markers including, but not limited to, IL-1α, IL-1β, IL-2, IL-4, IL-6, IL-8, IL-10, IL-12 p40, IL-12 p70, IL-17A, IFN- [symbol] , TNF-α, GM-CSF. Frozen plasma samples were thawed at room temperature (RT) and analyzed following MILLIPLEX® protocol guidelines. Cytokine concentrations were measured using a Bio-Plex® 200 (Bio-Rad®, Hercules, Calif.) and Bio-Plex® Manager™ software (Bio-Rad®, Hercules, Calif.). Sample volume was doubled to ensure measurable levels of cytokines, and assay output data was adjusted to reflect concentrations in plasma samples. Each multiplex assay was performed in duplicate, and cytokine levels were evaluated in three independent measurements.

### Data Analysis

Cytokine data gathered from the multiplex immunoassay were analyzed using data mining and predictive analytical methods. PCA was used to identify important cytokines by studying their contributions to each principal component (PC) as well as the associations between cytokines. Supervised machine learning models were created to determine whether cytokine levels can accurately predict which patients are more likely to experience mesh exposure post-surgery.

### Descriptive Analytics

The statistical programming language R (version 4.1.2) was used to analyze raw cytokine data values generated from the multiplex immunoassay. The imported data structure contained 60 observations (20 subjects×3 independent measurements) and 40 total variable fields (13 cytokines×3 blood treatments+1 target variable). The target variable was the subject outcome, which indicated post-surgical complication that subjects might have experienced following POP surgery. Observations marked ‘presence’ represent subjects who experienced mesh exposure through the vaginal wall. Observations marked ‘absence’ represent subjects who did not experience any mesh exposure through the vaginal wall. Univariate and multivariate methods were used to explore the data set, including identifying missing values, analyzing outliers, and visualizing frequency distributions.

### Principal Component Analysis

PCA was performed using the FactoMineR package (version 2.4) to identify associations between cytokines. Before analysis, data transformations were performed on each variable to correct for skewness in the distribution. The amount of skewness was calculated to assess the symmetry of distribution for each variable using Equation 1, where x̅ is the sample mean. Each cytokine's distribution was corrected for skewness using either a natural logarithm, square root, or inverse square root method. Using Equation 2, Zscore standardization was also applied to scale each cytokine variable, thus ensuring that the mean was equal to 0 and the standard deviation equal to 1. Bi-plots were created to visualize PCs with the highest degree of variance explained. The eigenvectors were overlayed on the bi-plots to visualize correlations and identify hidden patterns between cytokines.

$$skewness = \frac{\frac{1}{n} {\sum }_{i = 1}^{n} {( {x}_{i} - \overline{x} )}^{3}}{{[ \frac{1}{n - 1} {\sum }_{i = 1}^{n} {( {x}_{i} - \overline{x} )}^{2} ]}^{3 / 2}} \qquad Equation 1$$

$$Zscore = \frac{( {x}_{i} - \overline{x} )}{\sqrt{\frac{1}{n - 1} {\sum }_{i = 1}^{n} {( {x}_{i} - \overline{x} )}^{2}}} \qquad Equation 2$$

### Predictive Analytics

Supervised machine learning models were created using the Caret package (version 6.0-90) in the R programming language. The four models trained were Decision Tree, Logistic Regression, Naive Bayes, and Artificial Neural Network. This approach focused on the dataset from the experimental group only (cytokine expression for blood exposed to polypropylene mesh). The model was trained using 70% of data, while the remaining 30% of data was used to test the model. Each group contained an equal distribution of subjects who did or did not experience post-surgical mesh exposure through the vaginal wall—the prediction target for each model. The models were trained and tested to generate measures of accuracy, sensitivity, and specificity for the prediction of subjects to have post-surgical mesh exposure.

Additionally, this process was replicated to study the effects of reducing the number of cytokines needed to predict a post-surgical mesh exposure. A Random Forest algorithm was used to select important cytokines for this study. These models were trained and tested for accuracy, sensitivity, and specificity as detailed above. Results are compared to models trained with all 13 cytokines.

### Example 1

### Principal Component Analysis

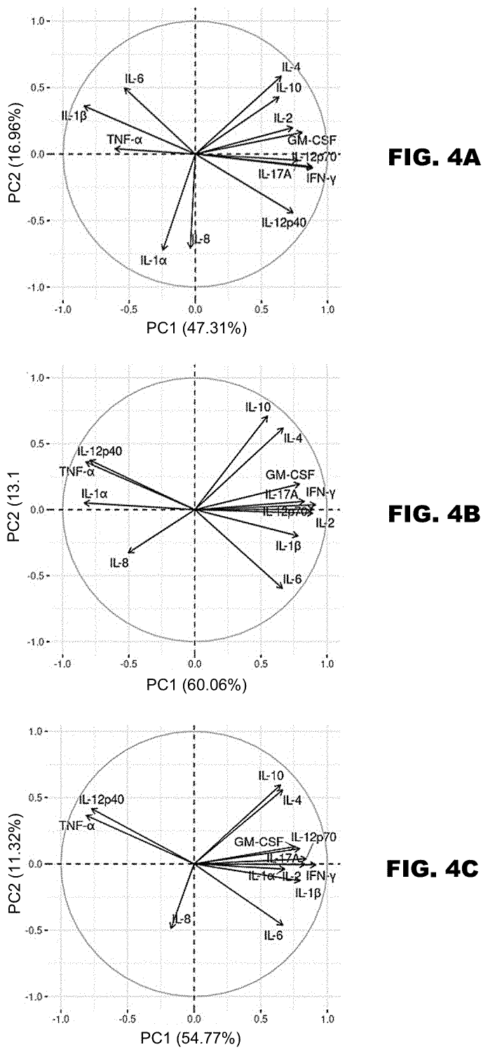

To identify significant associations among the cytokines, PCA was utilized to examine 60 blood samples (20 subjects×3 blood treatments). FIGS. 4A-4C depict bi-plots of each blood treatment and summarizes the intercorrelated relationships among individual inflammatory mediators. The combined variances explained for PC 1 and PC 2 in blood samples incubated with LPS (FIG. 4A), polypropylene mesh (FIG. 4B), or alone (FIG. 4C) were 64.27%, 73.16%, and 66.09%, respectively. In all three treatment groups, IL-10 and IL-4 align in the same directions, as do IL-12 p70 and IFN- [symbol] , indicating a positive correlation among these cytokines. In contrast, IL-6 and IL-12 p40 are negatively correlated when comparing stimulation of blood via LPS (FIG. 4A) vs. polypropylene mesh (FIG. 4B). Only IL-1α displays a negative correlation when comparing blood incubated with polypropylene mesh (FIG. 4B) vs. blood incubated alone (FIG. 4C).

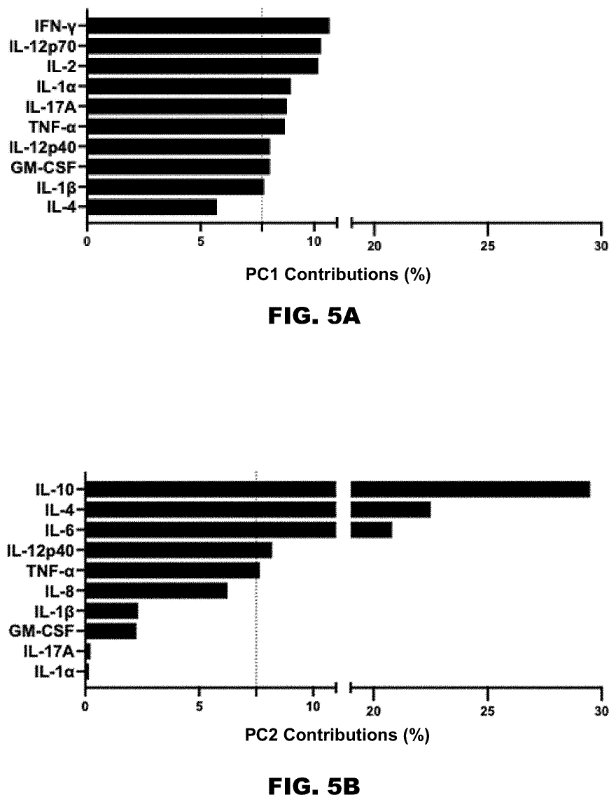

When PCA was utilized to examine only blood samples incubated with polypropylene mesh, PC 1 and PC 2 explained 60.1% and 13.1% of the total data variance, respectively (FIG. 4B). FIG. 5A displays each cytokine's contribution to PC 1 and illustrates that IFN- [symbol] , IL-12 p70, and IL-2 are the predominant contributors to the variance explained in PC 1. In addition, IL-1α, IL-17A, and TNF-α exhibited contributions above a level expected if the contribution were uniform. All other cytokines have contributions to PC 1 similar to or less than what would be expected if the contribution of all cytokines were uniform. FIG. 5B illustrates that the predominant contributors to the variance explained in PC 2 are IL-10, IL-4, and IL-6. All other cytokines have contributions to PC 2 similar to or less what would be expected if the contribution of all cytokines were uniform.

According to the present disclosure, the PCA analysis, as illustrated in FIGS. 4A-4C, reveals several significant associations among the cytokines. Several cytokines display positive correlations when comparing the two different stimuli, LPS and polypropylene mesh. However, IL-6 and IL-12 p40 are negatively correlated between these two treatments. Thus, these two cytokines may explain different inflammatory responses induced by LPS vs. polypropylene mesh. When comparing blood samples incubated alone to those incubated in the presence of polypropylene mesh, only IL-1 α exhibits a negative correlation, demonstrating that this pro-inflammatory mediator might be uniquely affected by the mesh stimulus. Furthermore, there were two groups of cytokines identified to be positively correlated (IL-10 and IL-4; IL-12 p70 and IFN-γ), indicating that one of the cytokines in each group could be eliminated to reduce the number of cytokines tested in a clinical setting.

The cytokines that contribute most to each PC are segregated into pro-and anti-inflammatory cytokines (FIGS. 5A-5B). When cytokine data from patient blood incubated with mesh were analyzed using PCA, cytokines IFN-γ, IL-12 p70, and IL-2 were the largest contributors to the variance explained in PC 1. These markers are identified as pro-inflammatory agents, which suggests that pro-inflammatory cytokines may heavily influence PC 1. In contrast, cytokines IL-10, IL-4, and IL-6 were the largest contributors to the variance explained in PC 2. IL-4 and IL-10 are prominent anti-inflammatory cytokines, suggesting that anti-inflammatory cytokines heavily influence PC 2. IL-6, previously thought to have pro-inflammatory function only, is recently recognized as potentially having both pro- and anti-inflammatory roles in COVID-19 and diabetes.

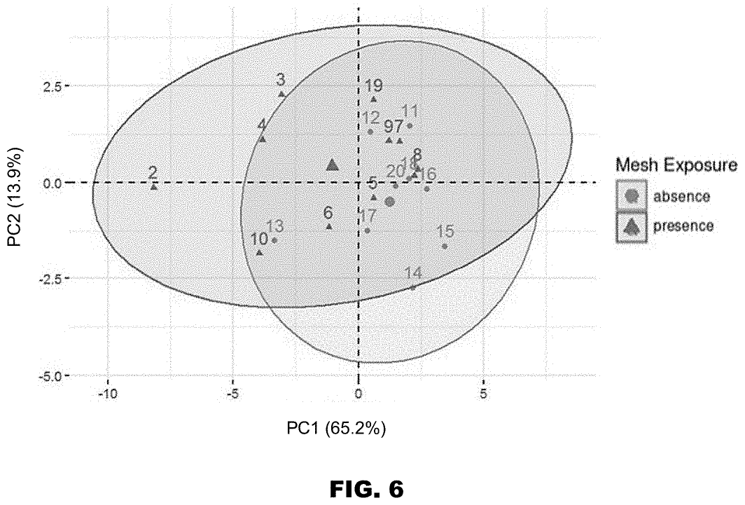

In order to visualize associations between the subjects presenting absence or presence of post-surgical mesh exposure, a bi-plot illustrating individual subjects was created (FIG. 6). This bi-plot reveals a high percentage of variability represented by the first two PCs (79.1%). Blood samples from subjects who did not experience post-surgical mesh exposure were heavily represented by positive PC 1 values, while blood samples from subjects with presence of post-surgical mesh exposure were generally represented by positive PC 2 values.

When juxtaposing the bi-plot of polypropylene-stimulated cytokine observations (FIG. 4B) with that of mesh exposure outcome (FIG. 6), it can be extrapolated that pro-inflammatory cytokines IL-12 p40, IL-1α, and TNF-α are positioned in the region of the bi-plot that uniquely corresponds to surgical outcomes with mesh exposure present in patients. Such juxtaposition suggests that IL-12 p40, IL-1α, and TNF-α may be associated with presence of post-surgical mesh exposure. These observations may inspire potential therapeutic strategies that could improve post-surgical outcome. For example, surgical mesh could be designed to modulate these key pro-inflammatory cytokines. In this way, while supporting the pelvic structure, the mesh could simultaneously function in controlling the cytokine response to minimize biomaterial rejection.

To examine predictive capabilities of cytokine level measurements in supervised machine learning models, four supervised learning models incorporating all 13 cytokines were trained using 70% of the available data; the remaining 30% was used to test the models' accuracy when predicting the presence of mesh exposure. All four learning machines achieved at least 62% training accuracy (Table 1). Artificial Neural Network achieved the highest prediction accuracy of 83.3%, while Decision Tree and Naïve Bayes both achieved a prediction accuracy of 61.1%. Naïve Bayes, Decision Tree, and Artificial Neural Network excelled at correctly predicting patients with presence of mesh exposure post-surgery (88.9%). Artificial Neural Network was superior for correctly predicting patients who did not experience mesh exposure post-surgery (77.8%).

Table 1 describes the accuracy of supervised machine learning models and demonstrates that cytokines exhibit predictive capabilities. Previous studies have performed predictive analysis for POP using risk factors derived from patient medical history; however, such data can be incomplete and inaccurate. The present disclosure demonstrates the utility that measured responses of biological samples can also have in developing robust predictive models. According to the present disclosure, the prediction accuracy for all four models exceeded 50%, with the Artificial Neural Network model demonstrating the best overall performance, predicting POP surgical outcomes with 83.3% accuracy when trained with all 13 cytokines. This predictive capability is similar to that reported for prediction derived from patient medical history, despite this study comprising a significantly smaller patient group. Considering the small population size, these results represent relatively high prediction accuracy for healthcare data.

TABLE 1
Summary of supervised learning model statistics. All 13 cytokines were utilized
to predict the presence or absence of post-surgical mesh exposure.

| | Training | Prediction | | | | | Prediction |
|---|---|---|---|---|---|---|---|
| Model | Accuracy | Accuracy | 95% CI | | Sensitivity | Specificity | Kappa |
| Artificial | 79.0% | 83.3% | 0.586 | 0.964 | 77.8% | 88.9% | 0.667 |
| Neural Network | | | | | | | |
| Decision Tree | 64.0% | 61.1% | 0.357 | 0.827 | 33.3% | 88.9% | 0.222 |
| Naïve Bayes | 62.3% | 61.1% | 0.357 | 0.827 | 33.3% | 88.9% | 0.222 |
| Logistic Reg | 72.5% | 50.0% | 0.260 | 0.740 | 55.5% | 44.4% | 0.000 |

### Example 2

### Predictive Analysis Using Feature Selection

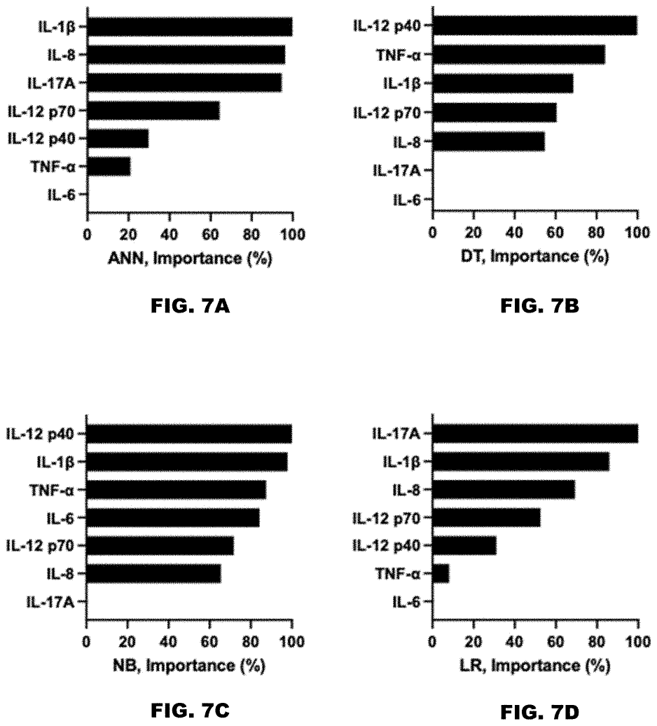

To explore whether a smaller set of cytokines could achieve similar predictive results, additional models were created and predictive analysis was performed. Feature selection using a Random Forest method identified a group of 7 cytokines capable of yielding effective predictive analysis: IL-1β, IL-8, IL-12 p40, IL-12 p70, TNF-α, IL-17A, and IL-6. FIGS. 7A-7D illustrate that models exhibited variation amongst the importance of cytokines when implementing this more targeted group of cytokines. IL-1β and IL-8 are strongly represented in all models, while IL-6 is important in only Naïve Bayes.

TABLE 2
Summary of supervised learning model statistics. Feature selection via
Random Forest was used to identify a group of 7 cytokines capable of yielding
effective predictive analysis. The subset of cytokines was utilized to
predict the presence or absence of post-surgical mesh exposure.

| | Training | Prediction | | | | | Prediction |
|---|---|---|---|---|---|---|---|
| Model | Accuracy | Accuracy | 95% CI | | Sensitivity | Specificity | Kappa |
| Artificial | 80.7% | 77.8% | 0.524 | 0.936 | 66.7% | 88.9% | 0.556 |
| Neural Network | | | | | | | |
| Decision Tree | 64.0% | 61.1% | 0.356 | 0.827 | 33.3% | 88.9% | 0.222 |
| Naïve Bayes | 71.5% | 55.6% | 0.308 | 0.785 | 33.3% | 77.8% | 0.111 |
| Logistic Reg | 80.5% | 72.2% | 0.465 | 0.903 | 66.7% | 77.8% | 0.444 |

Table 2 illustrates that all models achieved at least 64% training accuracy. The Logistic Regression model that employed the 7 selected cytokines outperformed in prediction accuracy (72.7%), training accuracy (80.5%), sensitivity (66.7%), and specificity (77.8%) compared to the Logistic Regression model that incorporated all of the cytokine data. Moreover, Decision Tree models achieved the same result when employing the selected cytokines or when all cytokines were included. The prediction accuracy in Naïve Bayes and Artificial Neural Network models executed with the 7 selected cytokines decreased slightly by 9% and 6.6%, respectively, compared to the same models that used all of the cytokine data.

When creating models trained with a subgroup of 7 cytokines (Table 2) and selected using a Random Forest method, the Artificial Neural Network model maintained the greatest effectiveness with respect to sensitivity, specificity, and prediction accuracy. Moreover, the group of selected cytokines outperformed the larger group of cytokines in the Logistic Regression model and achieved the same results in the Decision Tree model. The Naïve Bayes and Artificial Neural Network prediction accuracy dropped less than 10% when using the subgroup of cytokines, thus demonstrating the resiliency of these models. These results demonstrate that the predictive capabilities are retained with fewer cytokines, which would enhance clinical feasibility by reducing the cost and time associated with this clinical decision tool.

The preceding description is exemplary in nature and is not intended to limit the scope, applicability, or configuration of the disclosure in any way. Various changes to the described embodiments may be made in the function and arrangement of the elements described herein without departing from the scope of the disclosure.

Unless defined otherwise, all technical and scientific terms used herein have the same meaning as commonly understood by one of ordinary skill in the art to which this invention is related.

This written description uses examples to disclose the present disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only and is not intended to limit the invention so further described in such appended claims.

What is claimed:

1. A method of evaluating a subject's risk of one or more complications associated with pelvic organ prolapse surgery, the method comprising:
obtaining, by a computing system comprising one or more computing devices, sample data associated with the subject;
inputting, by the computing system, the sample data into a machine-learned immune response model;
receiving, by the computing system as an output of the machine-learned immune response model, one or more predictions of post-surgical complications of mesh exposure through the vaginal wall associated with the subject; and
performing a pelvic organ prolapse repair surgery on the subject, wherein the surgery is performed based at least in part on the one or more predictions of post-surgical complications by the machine-learned immune response model associated with a likelihood of success.

2. The method of claim 1, wherein the sample data comprises immune response marker data.

3. The method of claim 1, wherein the output comprises an expression visualization that visualizes the predicted post-surgical outcome associated with the subject; and
the method further comprises displaying, by the computing system, the visualization on a display screen.

4. The method of claim 1, further comprising: generating, by the computing system based at least in part on the predicted post-surgical outcome output by the machine-learned immune response model, a treatment guide that describes the subject's likelihood of success for the pelvic organ prolapse surgery.

5. The method of claim 4, further comprising: displaying, by the computing system, the treatment guide on a display screen.

6. The method of claim 2, wherein the immune response marker comprises a pro-inflammatory cytokine, an anti-inflammatory cytokine, a Treg cytokine, a tumor necrosis factor (TNF) superfamily protein, an interferon (IFN) family protein, a T helper 17 (Th17) immunity related gene, a matrix metalloproteinase (MMP), or a combination thereof.

7. The method of claim 2, wherein the immune response marker data comprises IL-1α, IL-1β, IL-2, IL-4, IL-6, IL-8, IL-10, IL-11, IL-12 p40, IL-12 p70, IL-17A, IL-17F, IL-21, IL-19, IL-20, IL-22, IL-23, IL-25, IL-26, IL-27 p28, IL-28A/IFN-I2, IL-29/IFN-I1, IL-31, IL-32, IL-33, IL-34, IL-35, sIL-6Ra, pg130/sIL-6Rβ, IFN-a2, IFN-β, IFN- [symbol] , TNF-α, sTNF-R1, sTNF-R2, TWEAK/TNFSF12, APRIL/TNFSF13, BAFF/TNFSF13B, LIGHT/TNFSF14, TSLP, GM-CSF, sCD40L, sCD163, sCD30/TNFRSF8, Chitinase-3-like 1, MMP-1, MMP-2, MMP-3, Osteocalcin, Osteopontin, Pentraxin-3, or a combination thereof.

8. A computing system, the computing system comprising:
a machine-learned immune response model trained with training data;
one or more processors; and
one or more non-transitory computer-readable media that store instructions that, when executed by the one or more processors, cause the one or more processors to perform operations, the operations comprising:
obtaining sample data associated with a subject, wherein the data comprises at least one set of sample data associated with the subject;
inputting the sample data into the machine-learned immune response model;
receiving, as an output of the machine-learned immune response model, one or more predictions of surgical outcomes associated with the subject.

9. The computing system of claim 8, wherein the sample data comprises a plurality of immune response marker data.

10. The computing system of claim 8, wherein the sample data associated with the subject comprises a blood sample collected from the subject.

11. The computing system of claim 8, wherein the training data comprises one or more sets of sample data.

12. The computing system of claim 11, wherein the biometric data comprises a plurality of immune response markers present in the data sample of the subject.

13. The computing system of claim 8, wherein:
the output comprises an immune response model visualization that visualizes the one or more expression patterns of immune markers associated with the subject;
and the operations further comprise displaying the visualization on a display screen.

14. The computing system of claim 8, wherein the operations further comprise:
generating, based at least in part on the predicted post-surgical outcome output by the machine-learned immune response model, a treatment guide that describes the subject's likelihood of success for the pelvic organ prolapse repair surgery.

15. The computing system of claim 13, wherein the operations further comprise:
displaying the treatment guide on a display screen.

16. The computing system of claim 8, wherein the machine-learned immune response model comprises one or more of: an artificial neural network, a support vector machine, a decision tree, a naïve bayes, or a logistic regression.

17. The computing system of claim 8, wherein the one or more tangible, non-transitory computer-readable media cause the one or more processors to perform additional operations, the additional operations comprising:
evaluating an objective function that evaluates a difference between the measured immune marker expression for each set of patient sample data and the immune response model for such set of patient sample data; and
adjusting one or more parameters of the machine-learned immune response model to improve the objective function.

18. The computing system of claim 9, wherein the immune response marker data comprises a pro-inflammatory cytokine, an anti-inflammatory cytokine, a Treg cytokine, a tumor necrosis factor (TNF) superfamily protein, an interferons (IFN) family protein, a T helper 17 (Th17) immunity related gene, a matrix metalloproteinase (MMP), or a combination thereof.

19. The computing system of claim 9, wherein the immune response marker data comprises IL-1α, IL-1β, IL-2, IL-4, IL-6, IL-8, IL-10, IL-11, IL-12 p40, IL-12 p70, IL-17A, IL-17F, IL-21, IL-19, IL-20, IL-22, IL-23, IL-25, IL-26, IL-27 p28, IL-28A/IFN-I2, IL-29/IFN-I1, IL-31, IL-32, IL-33, IL-34, IL-35, sIL-6Ra, pg130/sIL-6Rβ, IFN-a2, IFN-β, IFN- [symbol] , TNF-α, sTNF-R1, sTNF-R2, TWEAK/TNFSF12, APRIL/TNFSF13, BAFF/TNFSF13B, LIGHT/TNFSF14, TSLP, GM-CSF, sCD40L, sCD163, sCD30/TNFRSF8, Chitinase-3-like 1, MMP-1, MMP-2, MMP-3, Osteocalcin, Osteopontin, Pentraxin-3, or a combination thereof.
